(19) United States
(12) Patent Application Publication
(10) Pub. No.: US 2023/0062463 A1
(43) Pub. Date: Mar. 2, 2023

(54) STORAGE MEDIUM, DISPLAY CONTROL DEVICE, AND MEDICAL IMAGE DISPLAY SYSTEM

(71) Applicant: Konica Minolta, Inc., Tokyo (JP)

(72) Inventor: Yasuhiko Nishida, Kawasaki-shi (JP)

(73) Assignee: Konica Minolta, Inc., Tokyo (JP)

(21) Appl. No.: 17/819,637

(22) Filed: Aug. 15, 2022

(30) Foreign Application Priority Data
Sep. 2, 2021 (JP) 2021-143271

(51) Int. Cl. G16H 30/40; A61B 5/00; G16H 10/60; G16H 30/20

(52) U.S. Cl. CPC G16H 30/40; A61B 5/7425; G16H 10/60; G16H 30/20

(57) ABSTRACT

A non-transitory computer readable storage medium storing instructions that cause a computer of a display control device to: determine whether to display, in addition to a medical image of a first patient, a medical image of a second patient on a display; and upon determining to display the medical image of the second patient, conceal at least one or a plurality of pieces of patient information of the second patient to be shown in the medical image of the second patient.

### CROSS-REFERENCE TO RELATED APPLICATIONS

The entire disclosure of Japanese Patent Application No. 2021-143271 filed on Sep. 2, 2021 is incorporated herein by reference.

### BACKGROUND

### Technical Field

The present invention relates to a storage medium, a display control device, and a medical image display system.

### Description of Related Art

Medical images that are obtained by computed tomography (CT), computed radiography (CR), magnetic resonance imaging (MRI), a mammography apparatus, an ultrasonic diagnosis apparatus, etc., have conventionally been displayed on monitors or been printed so that doctors can make a diagnosis. In many cases, doctors explain a condition to a patient by showing a medical image, in order to explain the purpose and the content of medical examination and treatment to the patient and obtain understanding (informed consent) of the patient before starting treatment.

Meanwhile, hospitals and clinics must prevent personal information of patients from leaking, in order to protect it. For example, medical image diagnosis apparatuses that output medical image data together with patient information have been developed. One medical image diagnosis apparatus performs displaying and outputting while not showing or masking patient information that is not permitted to be disclosed, based on preliminarily set data that shows whether disclosure of each item of patient information is permitted for each output destination such as a monitor, a printer, a storage medium, or an image server (refer to JP 2005-296333A).

In a situation in which a doctor explains a condition to a patient to be treated by showing a medical image, as described above, the condition may be explained by showing a medical image of a patient other than the patient to be treated (another patient). In such a case, patient information of the another patient is not concealed but is shown in the medical image of the another patient. Thus, unfortunately, the patient to be treated can see the patient information of the another patient, and privacy of the another patient cannot be protected.

The invention disclosed in JP 2005-296333A does not assume the case in which a patient to be treated can look at a medical image of another patient.

### SUMMARY

One or more embodiments of the present invention provide a technological improvement over conventional storage mediums, display control devices, and medical image display systems. For example, one or more embodiments provide a storage medium storing instructions, a display control device, and a medical image display system that enable protecting privacy of a patient other than a patient to be treated, in displaying a medical image, which provides a practical and technological improvement over conventional storage mediums, display control devices, and medical image display systems.

According to an aspect of the present invention, a non-transitory computer readable storage medium stores instructions configured to cause a computer to function as the following units, the computer being provided to a display control device that is configured to display a medical image of a first patient and a medical image of a second patient on a display, the units including: a determination unit configured to determine whether to permit displaying the medical image of the second patient on the display in addition to the medical image of the first patient; and a concealment unit configured to conceal at least one or a plurality of pieces of patient information of the second patient to be shown in the medical image of the second patient, in response to the determination unit determining to permit displaying the medical image of the second patient.

According to another aspect of the present invention, a non-transitory computer readable storage medium stores instructions configured to cause a computer to function as the following units, the computer being provided to a display control device that is configured to display a medical image of a first patient and a medical image of a second patient on a display, the units including: a determination unit configured to determine whether to permit displaying the medical image of the second patient on the display in addition to the medical image of the first patient; and a hiding unit configured to hide at least one or a plurality of pieces of patient information of the second patient to be shown in the medical image of the second patient, in response to the determination unit determining to permit displaying the medical image of the second patient.

According to another aspect of the present invention, a display control device is configured to display a medical image of a first patient and a medical image of a second patient on a display, the display control device including a hardware processor configured to: determine whether to permit displaying the medical image of the second patient on the display in addition to the medical image of the first patient; and conceal at least one or a plurality of pieces of patient information of the second patient to be shown in the medical image of the second patient, in response to determining to permit displaying the medical image of the second patient.

According to another aspect of the present invention, a display control device is configured to display a medical image of a first patient and a medical image of a second patient on a display, the display control device including a hardware processor configured to: determine whether to permit displaying the medical image of the second patient on the display in addition to the medical image of the first patient; and hide at least one or a plurality of pieces of patient information of the second patient to be shown in the medical image of the second patient, in response to determining to permit displaying the medical image of the second patient.

According to another aspect of the present invention, a medical image display system includes: any one of the above display control devices; a display device including a display; and a medical image management device configured to store image data of medical images and manage the image data.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram illustrating an example of a medical image display system according to one or more embodiments of the present invention;

FIG. 2 is a block diagram illustrating an example of a medical image display control device that is provided to the medical image display system according to one or more embodiments;

FIG. 3 illustrates an example of a concealment setting image;

FIG. 4 is a flowchart of a patient information concealment process;

FIG. 5 illustrates an example of showing medical images of a patient to be treated and another patient on a diagnostic reading screen;

FIG. 6 illustrates an example of showing a medical image of a patient to be treated on a diagnostic reading screen and showing a medical image of another patient on a comparative diagnostic reading screen;

FIG. 7 illustrates an example of a non-display setting screen of a third modification example;

FIG. 8 is a flowchart of a patient information hiding process of the third modification example;

FIG. 9A illustrates an example of showing medical images of a patient to be treated and another patient on a diagnostic reading screen of the third modification example;

FIG. 9B illustrates an example of showing a medical image of a patient to be treated on a diagnostic reading screen and showing a medical image of another patient on a comparative diagnostic reading screen of the third modification example;

FIG. 10A illustrates an example of showing medical images of a patient to be treated and another patient on a diagnostic reading screen of the third modification example; and

FIG. 10B illustrates an example of showing a medical image of a patient to be treated on a diagnostic reading screen and showing a medical image of another patient on a comparative diagnostic reading screen of the third modification example.

### DETAILED DESCRIPTION OF EMBODIMENTS

Hereinafter, embodiments of the present invention will be described with reference to the drawings. Note that the technical scope of the present invention should not be limited to the following embodiments and to the illustrated examples.

<1. Overview of Medical Image Display System>

First, an overview of a medical image display system (hereinafter, a “system 100”) according to one or more embodiments will be described.

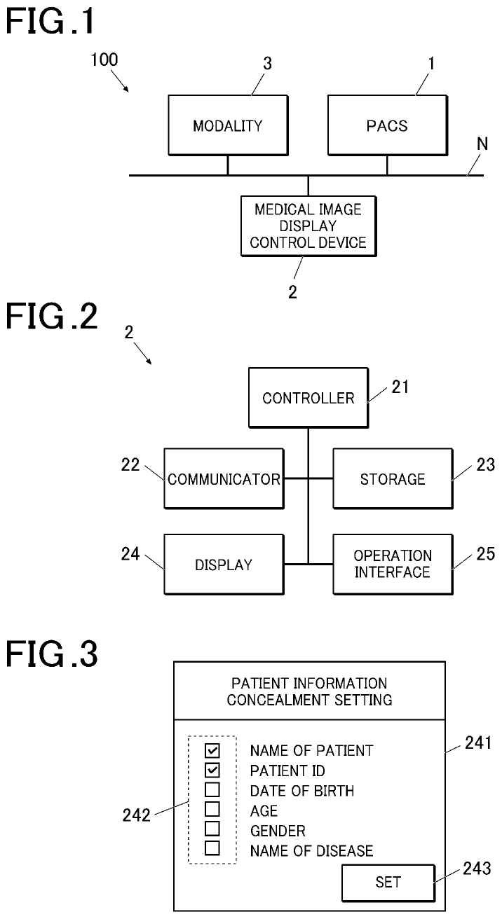

FIG. 1 is a block diagram illustrating an example of the system 100.

1-1. Schematic Configuration of Medical Image Display System

The system 100 includes, for example, a picture archiving and communication system (hereinafter, a “PACS 1”) as a medical image management device, and a medical image display control device (hereinafter, a “display control device 2”), as illustrated in FIG. 1.

The system 100 of one or more embodiments also includes a modality 3.

The devices 1 to 3 are able to communicate with each other via, for example, a communication network “N”, such as a local area network (LAN), a wide area network (WAN), or the Internet.

The system 100 may be able to communicate with a hospital information system (HIS), a radiology information system (RIS), or the like, which are not illustrated.

(Modality)

The modality 3 photographs a subject (a diagnosis target region of an examinee) and generates digital data of an image containing the subject (medical image) (hereinafter, “image data”).

The modality 3 includes, for example, a flat panel detector (FPD), a computed tomography (CT) device, a magnetic resonance imaging (MRI) device, and an ultrasonic diagnosis apparatus.

The modality 3 of one or more embodiments is configured to generate a medical moving image that is obtained by moving image photographing, in addition to a medical still image that is obtained by still image photographing.

Herein, the “moving image” represents a plurality of still images that, when being consecutively displayed at high speed, enable a viewer to perceive that the object in the still images continuously moves.

The moving image photographing of the present invention includes kymography, and the moving image includes dynamic images.

Kymography involves repeatedly emitting pulsed radiation (e.g., an X-ray) at a predetermined frame rate, or continuously emitting radiation of a low dose for a predetermined photographing time, to a subject, and it thereby provides a plurality of frames.

The dynamic images represent a series of frames that are obtained by kymography.

Such dynamic images make it possible to observe change with time of a subject (a diagnosis target region that moves, such as the lungs or the heart).

The modality 3 is in conformity with DICOM standard and generates image data in DICOM file format.

The image data in DICOM file format contains an image part and a header part.

Actual data of a medical image is written in the image part.

Various pieces of accompanying information are written in the header part.

The accompanying information is information attached to a medical image.

The accompanying information of one or more embodiments includes patient information, inspection information, series information, image information, grayscale softcopy presentation state (GSPS) information, and overlay information.

The patient information includes, for example, a patient ID, a patient name, a date of birth, age, gender, height, and weight.

The inspection information includes, for example, an inspection ID, a date of inspection, time of inspection, inspection description, a number of series, and a number of images.

The series information includes, for example, modality that is used, photographed region, photographing method (e.g., a photographing direction such as a slice direction, and use of a contrast medium), series description, laterality, a series number, a number of images, and an AE title.

The image information includes, for example, an image number, a patient direction, an image type, a pixel structure, longitudinal width, lateral width, tube voltage, mA, tube current, pixel spacing, slice thickness, slice position, a frame number, a number of frames, TR, TE, and WW/WC.

The modality 3 may include a console (not illustrated) that sets various photographing conditions and controls operation of each device of the modality 3.

In addition, the modality 3 may be configured to be installed in a photographing room or be movable (as an instrument carriage).

(Picture Archiving and Communication System)

The PACS 1 archives medical images obtained from the modality 3.

The PACS 1 is composed of a PC, a dedicated device, and so on.

The PACS 1 of one or more embodiments archives image data of a plurality of medical images in the form of a database, together with corresponding inspection information.

The PACS 1 may generate a thumbnail of a corresponding medical image based on image data of the medical image that is archived.

(Medical Image Display Control Device)

The display control device 2 controls to display a medical image that is obtained from the PACS 1.

The display control device 2 is composed of a PC in which a dedicated program is installed, a dedicated device, and so on.

The display control device 2 of one or more embodiments includes a display.

The display control device 2 of one or more embodiments displays a medical image on the display.

That is, the display control device 2 of one or more embodiments also functions as a display device.

The display control device 2 may display a medical image that is directly obtained from the modality 3 (without using the PACS 1).

Details of the display control device 2 will be described later.

1-2. Flow of Diagnosis Using Medical Image Display System

Diagnosis using the system 100 thus configured is conducted in accordance with the flow described below.

First, in response to a user photographing a subject by using the modality 3, the modality 3 generates image data of a medical image that contains the subject and transmits it to the PACS 1.

Upon receiving the image data from the modality 3, the PACS 1 archives the received image data.

In addition, upon receiving a signal that requests image data, from the display control device 2 or the like, the PACS 1 transmits corresponding image data to the display control device 2.

The display control device 2 that has obtained the image data from the PACS 1 displays a medical image based on the obtained image data.

A doctor reads the medical image that is displayed on the display control device 2 and makes a diagnosis of an examinee.

1-3. Modification Example of Medical Image Display System

The system 100 including the PACS 1, the display control device 2, and the modality 3 is described above; however, in the case in which at least one of the PACS 1 and the modality 3 includes a display, one or both of the PACS 1 and the modality 3 may have one or a plurality of functions of the display control device 2.

In this case, it is not necessary for the system 100 to include the display control device 2. In addition, one or both of the display control device 2 and the modality 3 may have one or a plurality of functions of the PACS 1.

In this case, it is not necessary for the system 100 to include the PACS 1.

The system 100 may also include an analysis device that analyzes a medical image (in particular, a dynamic image).

In this case, the PACS 1 may archive an analyzed image that is obtained from the analysis device.

In addition, in this case, the display control device 2 may control to display an analyzed image that is obtained from the PACS 1 or the analysis device.

<2. Details of Medical Image Display Control Device>

Next, details of the display control device 2, which is provided to the system 100, will be described.

FIG. 2 is a block diagram illustrating the display control device 2.

2-1. Configuration of Medical Image Display Control Device

The display control device 2 includes, for example, a controller 21 (hardware processor) and a communicator 22, as illustrated in FIG. 2.

The display control device 2 of one or more embodiments also includes a storage 23, a display 24 as described above, and an operation interface 25.

The devices 21 to 25 are electrically connected to each other via a bus or the like.

The controller 21 is composed of a CPU, a RAM, a ROM, and so on.

The ROM stores various instructions (e.g., programs) that are executed by the CPU, parameters necessary to execute the instructions, etc.

The CPU reads the various instructions that are stored in the ROM and loads them into the RAM, and the CPU executes various processes in accordance with the loaded instructions to control operation of each device of the display control device 2 in a centralized manner.

The ROM may be configured to be rewritable (allow update of instructions).

Before displaying a medical image of a patient to be treated (first patient) on the display 24, the controller 21 determines whether to permit displaying a medical image of another patient other than the patient to be treated (second patient). Herein, the controller 21 functions as a determination unit.

In the case in which the determination unit determines to permit displaying a medical image of the second patient, the controller 21 conceals at least one or a plurality of pieces of patient information of the second patient to be shown in the medical image of the second patient. Herein, the controller 21 functions as a concealment unit.

The controller 21 obtains pieces of patient information to be concealed, from among pieces of patient information of the second patient to be shown in the medical image of the second patient. Herein, the controller 21 functions as a first obtaining unit.

The communicator 22 is composed of a communication module and so on.

The communicator 22 transmits and receives various signals and various data with another device (such as the PACS 1, the modality 3, or the analysis device) that is connected wired or wirelessly via the communication network “N”.

The storage 23 is composed of a non-volatile memory, a hard disk drive, and so on.

The storage 23 stores image data of medical images.

The storage 23 also stores another patient mode setting information, viewing mode setting information, and concealment setting information, which will be described later.

The another patient mode setting information is information that specifies which of an another patient ON mode and an another patient OFF mode is set.

The viewing mode setting information is information that specifies which of a doctor viewing mode and a patient viewing mode is set.

The concealment setting information is information that specifies items of patient information of another patient to be concealed, in displaying a medical image of a patient other than a patient to be treated (another patient).

The display 24 is composed of, for example, a liquid crystal display (LCD), a cathode ray tube (CRT), or the like.

The display 24 displays a medical image in accordance with an image signal that is received from the controller 21, and so on.

The operation interface 25 is composed of a keyboard (such as cursor keys, numeric keys, and various function keys), a pointing device (a mouse or the like), a touch panel that is laminated on the surface of the display 24, and so on.

The operation interface 25 outputs a control signal to the controller 21 in accordance with operation performed by a user.

The display control device 2 including the display 24 and the operation interface 25 is described above; however, the display control device 2 may not include the display 24 and may output an image signal to another device (may control to display a medical image on another device) that is provided separately therefrom (such as a dedicated monitor, the PACS 1, or the analysis device) via the communicator 22.

The display control device 2 may receive a control signal from another device that is provided separately therefrom (such as a dedicated input device, the PACS 1, or the analysis device) via the communicator 22.

<3. Operation of Medical Image Display System>

Next, operation of the system 100 will be described.

3-1. Another Patient Mode Setting Process

The controller 21 of the display control device 2 executes an another patient mode setting process in accordance with the instructions stored in the ROM, in response to a user, such as a doctor, inputting an instruction to execute the another patient mode setting process, for example, via the operation interface 25.

The another patient mode includes an another patient ON mode and an another patient OFF mode.

The another patient ON mode is a mode in which showing a medical image of another patient on a diagnostic reading screen or a comparative diagnostic reading screen is permitted, in the state in which a medical image of a patient to be treated is shown on a diagnostic reading screen for reading a medical image.

The another patient OFF mode is a mode in which showing a medical image of another patient on a diagnostic reading screen or a comparative diagnostic reading screen is forbidden, in the state in which a medical image of a patient to be treated is shown on a diagnostic reading screen.

In the another patient mode setting process, the controller 21 makes the display 24 display an another patient mode setting screen (not illustrated) for selectively inputting the another patient ON mode or the another patient OFF mode, to receive input of the another patient mode setting information. The controller 21 then stores the input another patient mode setting information in the storage 23 and performs setting in accordance with this information.

3-2. Viewing Mode Setting Process

The controller 21 of the display control device 2 executes a viewing mode setting process in accordance with the instructions stored in the ROM, in response to a user, such as a doctor, inputting an instruction to execute the viewing mode setting process, for example, via the operation interface 25.

The viewing mode includes a doctor viewing mode and a patient viewing mode.

The doctor viewing mode is a mode to allow a user who is entitled to view personal information of a patient, such as a doctor, to look at a medical image of a patient to be treated or of another patient.

The patient viewing mode is a mode that is used in a situation such that a doctor explains a condition to a patient to be treated by showing medical images of the patient to be treated and another patient, and this mode allows the patient to be treated to look at a medical image of another patient. In such a situation, it is necessary to prevent personal information of the another patient from leaking to the patient to be treated.

In the viewing mode setting process, the controller 21 makes the display 24 display a viewing mode setting screen (not illustrated) for selectively inputting the doctor viewing mode or the patient viewing mode, to receive input of the viewing mode setting information. The controller 21 then stores the input viewing mode setting information in the storage 23 and performs setting in accordance with this information.

3-3. Concealment Setting Process

The controller 21 of the display control device 2 executes a concealment setting process in accordance with the instructions stored in the ROM, in response to a user, such as a doctor, inputting an instruction to execute the concealment setting process, for example, via the operation interface 25.

In the concealment setting process, the controller 21 makes the display 24 display a concealment setting screen 241 (refer to FIG. 3) for inputting concealment setting information, to receive input of the concealment setting information. In the example illustrated in FIG. 3, a user inputs check marks in checkboxes 242 and presses a set button 243. In response to this, the controller 21 stores concealment setting information that instructs concealing the information of the items that are marked with the check marks, in the storage 23, and it performs setting in accordance with this information.

In the example illustrated in FIG. 3, the items of patient information of another patient that are set to be concealed are a name of patient and a patient ID. On the other hand, the items of patient information of another patient that are not set to be concealed are a date of birth, age, gender, and a name of disease.

Note that the items of patient information of another patient are not limited to the examples illustrated in FIG. 3 and may be other items related to another patient.

In addition, all items of patient information of another patient may be set to be concealed, or one or some of the items may be set to be concealed.

3-4. Patient Information Concealment Process

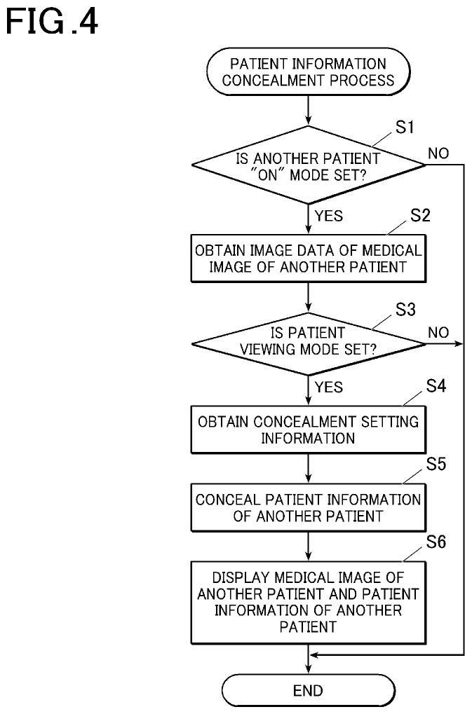

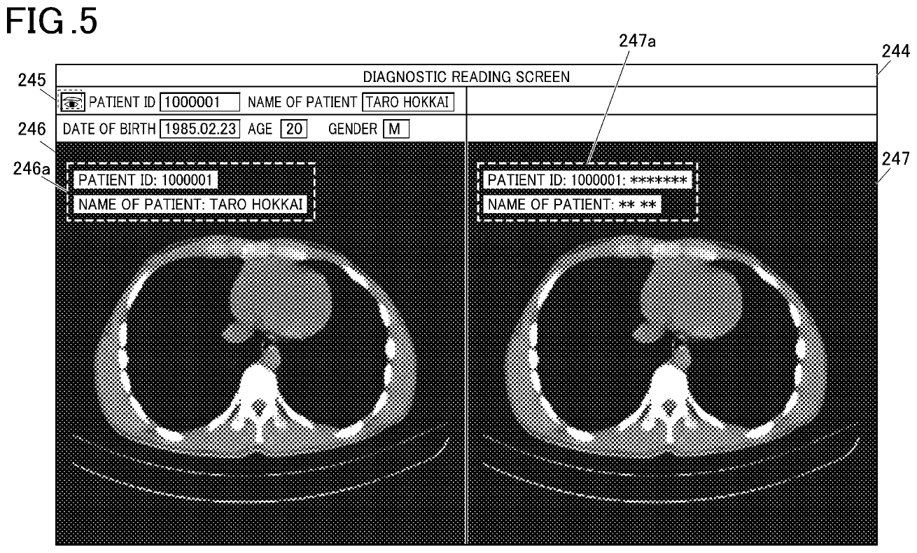

A doctor may show a medical image of a patient to be treated, on a diagnostic reading screen, for example, in a situation such that the doctor explains a condition to the patient to be treated by showing the medical image. At this time, the doctor or another user may input an instruction to display a medical image of another patient on the diagnostic reading screen, via the operation interface 25. In response to this, the controller 21 of the display control device 2 executes a patient information concealment process, which is illustrated in FIG. 4, in accordance with the instructions stored in the ROM.

In the patient information concealment process, patient information of the another patient is shown superimposed on a medical image of the another patient shown on the diagnostic reading screen. The displayed items of patient information of the another patient are preliminarily set (set as initial display settings).

In the patient information concealment process, the controller 21 obtains the another patient mode setting information from the storage 23 and determines whether the another patient ON mode is set (step S1). That is, before displaying a medical image of a first patient on the display 24, the controller 21 determines whether to permit displaying a medical image of a second patient.

In the case in which the another patient ON mode is set (step S1; YES), the controller 21 obtains image data of a medical image of another patient that the PACS 1 instructs to display (step S2).

Then, the controller 21 obtains the viewing mode setting information from the storage 23 and determines whether the patient viewing mode is set (step S3).

In the case in which the patient viewing mode is set (step S3; YES), the controller 21 obtains the concealment setting information from the storage 23 (step S4). That is, the controller 21 obtains pieces of patient information to be concealed (concealment setting information), from among pieces of patient information of the second patient to be shown in the medical image of the second patient.

Next, the controller 21 conceals the pieces of patient information that are set to be concealed, among the pieces of patient information contained in the image data obtained in step S2, based on the concealment setting information obtained in step S4 (step S5). That is, in the case in which the determination unit determines to permit displaying a medical image of the second patient, the controller 21 conceals at least one or a plurality of pieces of patient information of the second patient to be shown in the medical image of the second patient.

Thereafter, the controller 21 shows the pieces of patient information that are concealed in step S5, and the pieces of patient information that are obtained in step S2 and are not set to be concealed, in a manner superimposed on the medical image of the image data obtained in step S2, on the diagnostic reading screen on the display 24 (step S6). Then, the controller 21 terminates this process.

FIG. 5 illustrates an example of showing a medical image of another patient on a diagnostic reading screen 244 displayed on the display 24 in step S6 of the patient information concealment process.

In the example illustrated in FIG. 5, patient information of a patient to be treated (a patient ID of a patient to be treated, a name of a patient to be treated, and a date of birth, age, and gender of a patient to be treated) is shown in an information column 245 of a patient to be treated of the diagnostic reading screen 244. In addition, patient information 246a of the patient to be treated (a patient ID of a patient to be treated and a name of a patient to be treated) is shown superimposed on a medical image 246 of the patient to be treated, on the diagnostic reading screen 244.

Moreover, patient information 247a of another patient (a patient ID of another patient and a name of another patient) is shown superimposed on a medical image 247 of the another patient, on the diagnostic reading screen 244. Among the patient information 247a of the another patient in this case, a patient ID of the another patient and a name of the another patient are concealed and are shown by asterisks. Other pieces of patient information are not set as initial display settings and are thereby not shown irrespective of the concealment settings.

On the other hand, in the case in which the another patient ON mode is not set (step S1; NO), that is, the another patient OFF mode is set, or the patient viewing mode is not set (step S3; NO), that is, the doctor viewing mode is set, the controller 21 terminates this process.

### First Modification Example

Next, a first modification example of the above-described embodiments will be described.

The following description focuses on differences from the above-described embodiments.

The configuration of the system 100 of this modification example is the same as that of the system 100 of the above-described embodiments.

In this modification example, a doctor may display a medical image of a patient to be treated, on a diagnostic reading screen, for example, in a situation such that the doctor explains a condition to the patient to be treated by showing the medical image. At this time, the doctor or another user may input an instruction to display a medical image of another patient on a comparative diagnostic reading screen, via the operation interface 25. In response to this, the controller 21 of the display control device 2 executes the patient information concealment process.

In the patient information concealment process, patient information of the another patient is shown superimposed on a medical image of the another patient shown on the comparative diagnostic reading screen. In addition, the patient information of the another patient is shown in the another patient information column of the comparative diagnostic reading screen. The items of patient information of the another patient that are shown in the medical image of the another patient, and the items of patient information of the another patient that are shown in the another patient information column, are respectively preliminarily set (set as initial display settings).

In the patient information concealment process of this modification example, the controller 21 executes steps S1 to S5 of the patient information concealment process of the above-described embodiments.

In step S6, the controller 21 shows the pieces of patient information that are concealed in step S5, and the pieces of patient information that are obtained in step S2 and are not set to be concealed, in a manner superimposed on the medical image of the image data obtained in step S2, on the comparative diagnostic reading screen on the display 24. The controller 21 also shows the concealed pieces of patient information and the pieces of patient information that are not set to be concealed, in the another patient information column of the comparative diagnostic reading screen.

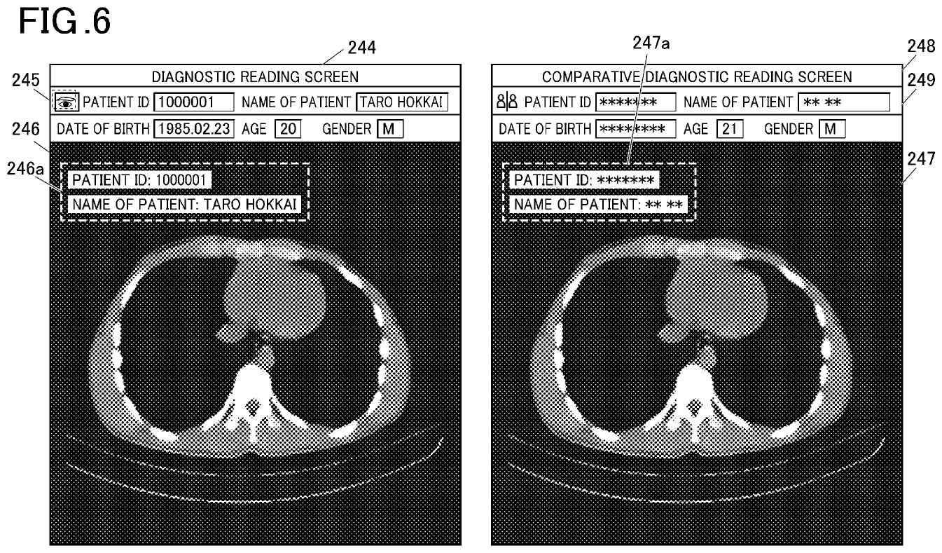

FIG. 6 illustrates an example of showing a medical image of another patient on a comparative diagnostic reading screen 248 displayed on the display 24 in step S6 of the patient information concealment process of this modification example.

In the example illustrated in FIG. 6, patient information of a patient to be treated is shown in the information column 245 of a patient to be treated of the diagnostic reading screen 244, as in the case of FIG. 5.

In addition, as in the case of FIG. 5, patient information 246a of the patient to be treated is shown superimposed on the medical image 246 of the patient to be treated, on the diagnostic reading screen 244.

On the other hand, patient information of another patient (a patient ID of another patient, a name of another patient, and a date of birth, age, and gender of another patient) is shown in an another patient information column 249 of the comparative diagnostic reading screen 248. Among the patient information of the another patient shown in the another patient information column 249, a patient ID, a name of the another patient, and a date of birth of the another patient are concealed and are shown by asterisks. The age and gender of the another patient are not set to be concealed and are shown as they are.

In addition, as in the case of FIG. 5, the patient information 247a of the another patient is shown superimposed on the medical image 247 of the another patient, on the comparative diagnostic reading screen 248. Among the patient information 247a of the another patient in this case, a patient ID of the another patient and a name of the another patient are concealed and are shown by asterisks. Other pieces of patient information are not set as initial display settings and are thereby not shown irrespective of the concealment settings.

### Second Modification Example

Next, a second modification example of the above-described embodiments will be described.

The following description focuses on differences from the above-described embodiments. The configuration of the system 100 of this modification example is the same as that of the system 100 of the above-described embodiments.

In the patient information concealment process of this modification example, the controller 21 executes steps S1 to S5 of the patient information concealment process of the above-described embodiments.

In step S6, the controller 21 embeds the pieces of patient information that are concealed in step S5, and the pieces of patient information that are obtained in step S2 and are not set to be concealed, in the medical image of the image data obtained in step S2. In the case in which an instruction is made to show a medical image of another patient on a diagnostic reading screen, the controller 21 shows a medical image of the another patient in which the patient information of the another patient is embedded, on the diagnostic reading screen on the display 24. On the other hand, in the case in which an instruction is made to show a medical image of another patient on a comparative diagnostic reading screen, the controller 21 shows a medical image of the another patient in which the patient information of the another patient is embedded, on the comparative diagnostic reading screen displayed on the display 24. The controller 21 also shows the patient information of the another patient, including concealed pieces of patient information of the another patient, in the another patient information column of the comparative diagnostic reading screen.

### Third Modification Example

Next, a third modification example of the above-described embodiments will be described.

The following description focuses on differences from the above-described embodiments. The configuration of the system 100 of this modification example is the same as that of the system 100 of the above-described embodiments.

In this modification example, in the case in which the determination unit determines to permit displaying a medical image of the second patient, the controller 21 hides at least one or a plurality of pieces of patient information of the second patient to be shown in the medical image of the second patient. Herein, the controller 21 functions as a hiding unit.

The controller 21 obtains pieces of patient information to be hidden, from among pieces of patient information of the second patient to be shown in the medical image of the second patient. Herein, the controller 21 functions as a second obtaining unit.

The storage 23 stores non-display setting information.

The non-display setting information is information that specifies the items of patient information of another patient to be hidden, in displaying a medical image of a patient other than a patient to be treated (another patient).

The controller 21 of the display control device 2 executes a non-display setting process in accordance with the instructions stored in the ROM, in response to a user, such as a doctor, inputting an instruction to execute the non-display setting process, for example, via the operation interface 25.

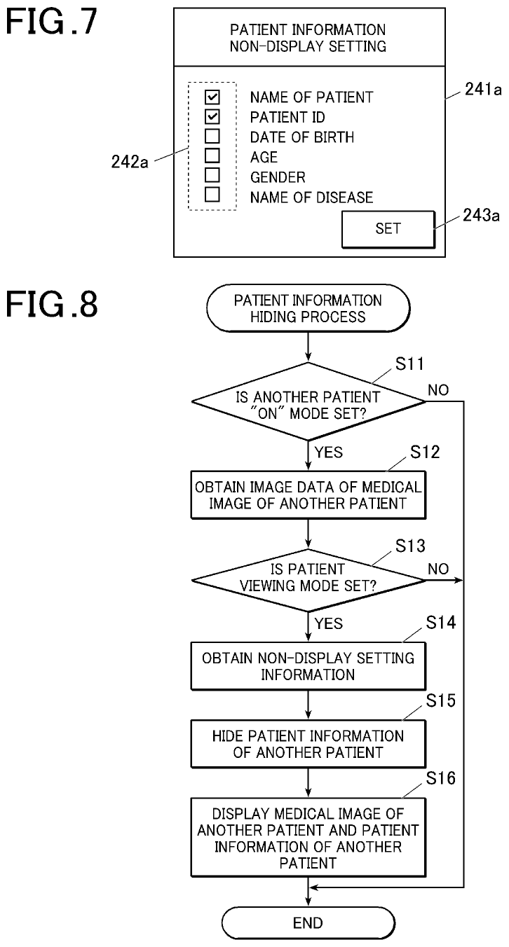

In the non-display setting process, the controller 21 makes the display 24 display a non-display setting screen 241a (refer to FIG. 7) for inputting non-display setting information, to receive input of the non-display setting information.

In the example illustrated in FIG. 7, a user inputs check marks in checkboxes 242a and presses a set button 243a. In response to this, the controller 21 stores non-display setting information that instructs hiding the information of the items that are marked with the check marks, in the storage 23, and it performs setting in accordance with this information.

In the example illustrated in FIG. 7, the items of the patient information of another patient that are set to be hidden are a name of patient and a patient ID. On the other hand, the items of the patient information of another patient that are not set to be hidden are a date of birth, age, gender, and a name of disease.

Note that the items of patient information of another patient are not limited to the examples illustrated in FIG. 7 and may be other items related to another patient.

In addition, all items of patient information of another patient may be set to be hidden, or one or some of the items may be set to be hidden.

FIG. 8 illustrates a flowchart of a patient information hiding process of this modification example.

In the patient information hiding process, patient information of another patient is shown superimposed on a medical image of the another patient shown on the diagnostic reading screen. The displayed items of patient information of the another patient are preliminarily set (set as initial display settings).

In the patient information hiding process of this modification example, the controller 21 executes steps S11 to S13 that are similar to steps S1 to S3 of the patient information concealment process of the above-described embodiments.

In the case in which the patient viewing mode is set (step S13; YES), the controller 21 obtains the non-display setting information from the storage 23 (step S14). That is, the controller 21 obtains pieces of patient information to be hidden, from among pieces of patient information of the second patient to be shown in the medical image of the second patient.

Next, the controller 21 hides the pieces of patient information that are set to be hidden, among the pieces of patient information contained in the image data obtained in step S12, based on the non-display setting information obtained in step S14 (step S15). That is, in the case in which the determination unit determines to permit displaying a medical image of the second patient, the controller 21 hides at least one or a plurality of pieces of patient information of the second patient to be shown in the medical image of the second patient.

Thereafter, the controller 21 shows the names of the items of patient information that are hidden in step S15, and the pieces of patient information that are obtained in step S12 and are not set to be hidden, in a manner superimposed on the medical image of the image data obtained in step S12, on the diagnostic reading screen on the display 24 (step S16). Then, the controller 21 terminates this process.

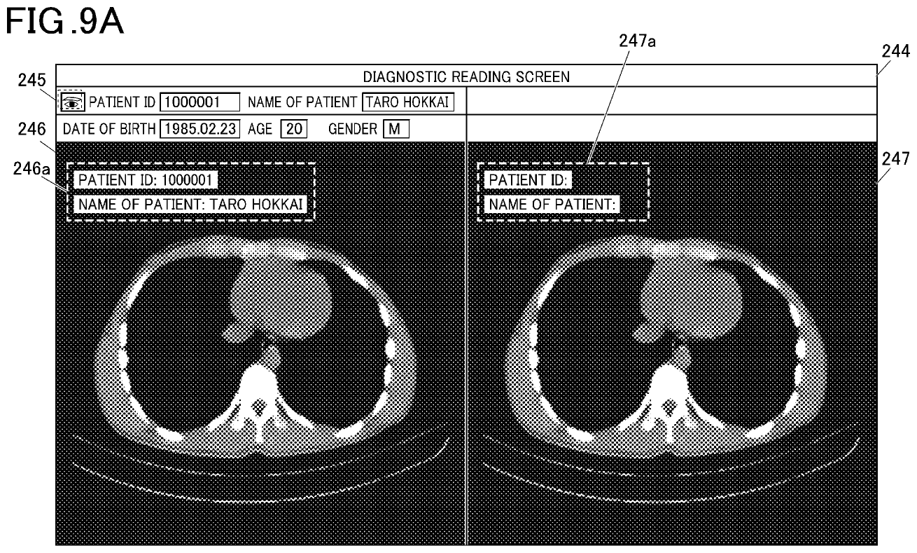

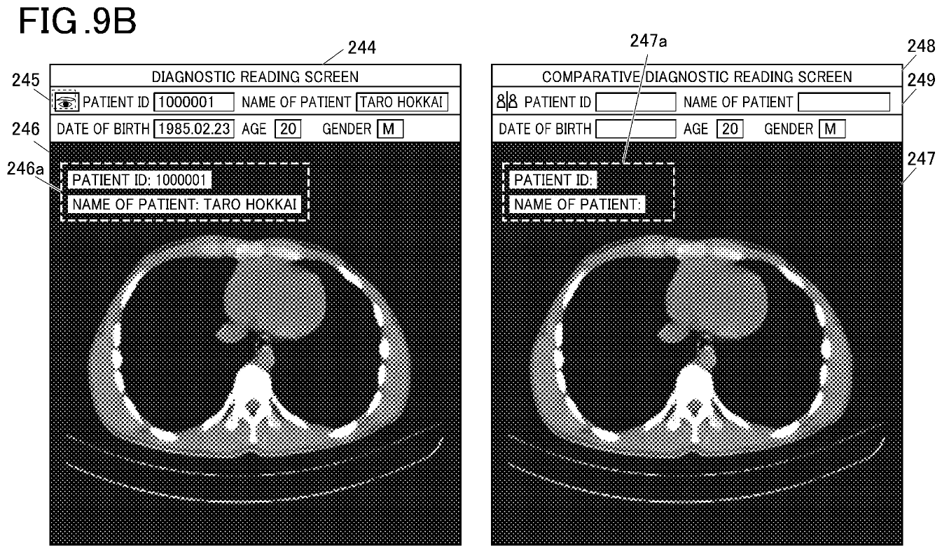

FIG. 9A illustrates an example of showing patient information 247a of another patient in a manner superimposed on a medical image 247 of the another patient on a diagnostic reading screen 244 of this modification example.

As illustrated in FIG. 9A, among the patient information 247a of the another patient, a patient ID of the another patient and a name of the another patient are set to be hidden, and only the names of the items are shown. Other pieces of patient information are not set as initial display settings and are thereby not shown irrespective of the non-display settings.

Moreover, a case in which an instruction is made to show a medical image of another patient on a comparative diagnostic reading screen will be described. In this case, patient information of another patient is shown superimposed on a medical image of another patient shown on a comparative diagnostic reading screen. In addition, patient information of the another patient is shown in the another patient information column of the comparative diagnostic reading screen. The items of patient information of the another patient that are shown in the medical image of the another patient, and the items of patient information of the another patient that are shown in the another patient information column, are respectively preliminarily set (set as initial display settings).

In this case, in step S16 of the patient information hiding process, the controller 21 shows the names of the items of patient information that are hidden in step S15, and the pieces of patient information that are obtained in step S12 and are not set to be hidden, in a manner superimposed on the medical image of the another patient, on the comparative diagnostic reading screen on the display 24. The controller 21 also shows only the pieces of patient information that are not set to be hidden, in the another patient information column of the comparative diagnostic reading screen.

FIG. 9B illustrates an example of showing patient information 247a of another patient in a manner superimposed on a medical image 247 of the another patient on a comparative diagnostic reading screen 248 of this modification example.

In the example illustrated in FIG. 9B, among the patient information 247a of the another patient, a patient ID of the another patient and a name of the another patient are set to be hidden, and only the names of the items are shown, as in the case of FIG. 9A. Other pieces of patient information are not set as initial display settings and are thereby not shown irrespective of the non-display settings.

In addition, in the example illustrated in FIG. 9B, a patient ID, a name, and a date of birth of the another patient are set to be hidden, and these pieces of information are not shown in the another patient information column 249. The age and gender of the another patient are not set to be hidden and are shown as they are.

The names of the items that are set to be hidden (e.g., a patient ID of another patient and a name of another patient) among the patient information 247a of the another patient may also be hidden.

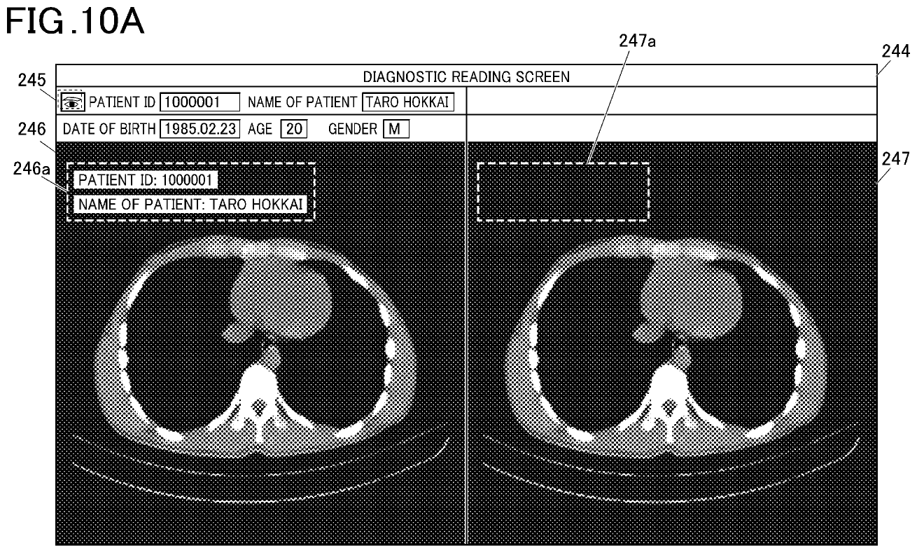

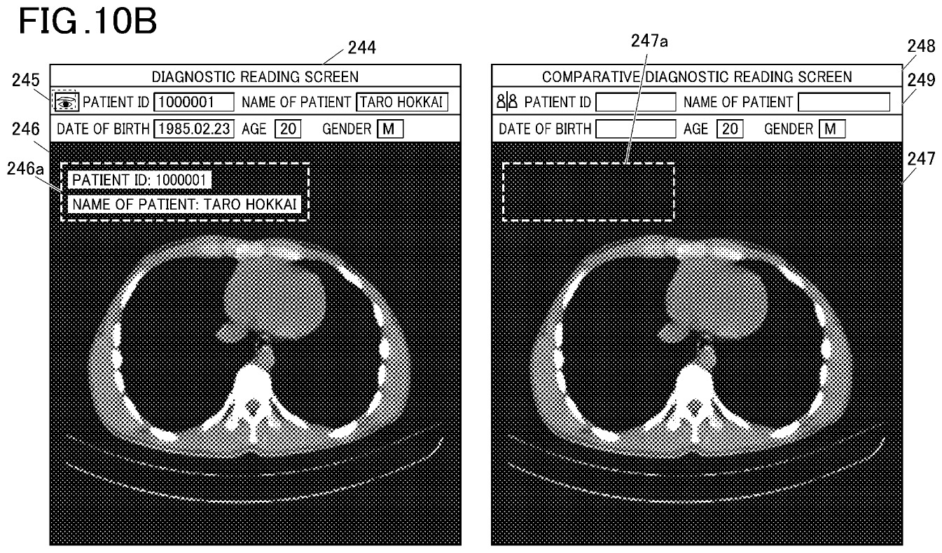

FIG. 10A illustrates an example of showing patient information 247a of another patient in which the names of the items are also set to be hidden, in a manner superimposed on a medical image 247 of the another patient on a diagnostic reading screen 244.

FIG. 10B illustrates an example of showing patient information 247a of another patient in which the names of the items are also set to be hidden, in a manner superimposed on a medical image 247 of the another patient on a comparative diagnostic reading screen 248.

In step S16 of the patient information hiding process of the third modification example, the controller 21 embeds the names of the items of patient information that are hidden in step S15, and the pieces of patient information that are obtained in step S12 and are not set to be hidden, in the medical image of the image data obtained in step S12. In the case in which an instruction is made to show a medical image of another patient on a diagnostic reading screen, the controller 21 shows a medical image of the another patient in which the patient information of the another patient is embedded, on the diagnostic reading screen on the display 24. On the other hand, in the case in which an instruction is made to show a medical image of another patient on a comparative diagnostic reading screen, the controller 21 may show a medical image of the another patient in which the patient information of the another patient is embedded, on the comparative diagnostic reading screen on the display 24. The controller 21 may also show pieces of the patient information of the another patient that are not set to be hidden, in the another patient information column of the comparative diagnostic reading screen.

<4. Advantageous Effects>

The display control device 2 described above is a display control device 2 configured to make the display 24 display a medical image of a first patient (a patient to be treated) and a medical image of a second patient (another patient who is a patient other than the patient to be treated). The display control device 2 includes the determination unit (controller 21) and the concealment unit (controller 21). The determination unit determines whether to permit displaying the medical image of the second patient on the display 24 in addition to the medical image of the first patient. The concealment unit conceals at least one or a plurality of pieces of patient information of the second patient to be shown in the medical image of the second patient in the case in which the determination unit determines to permit displaying the medical image of the second patient.

Thus, it is possible to provide a display control device that enables protecting privacy of a patient other than a patient to be treated, in displaying a medical image.

In the display control device 2, the concealment unit conceals at least one or a plurality of pieces of patient information of the second patient to be shown in a manner superimposed on the medical image of the second patient.

Thus, although patient information of another patient is shown superimposed (overlaid) on a medical image of the another patient, privacy of a patient other than a patient to be treated can be protected in displaying a medical image.

In the display control device 2, the concealment unit conceals at least one or a plurality of pieces of patient information of the second patient to be embedded in a medical image of the second patient.

Thus, although patient information of another patient is embedded in a medical image of the another patient, privacy of a patient other than a patient to be treated can be protected in displaying a medical image.

The display control device 2 also includes the first obtaining unit that obtains pieces of patient information to be concealed, from among pieces of patient information of the second patient to be shown in a medical image of the second patient.

With this configuration, items that are targets to protect privacy of patient information of a patient other than a patient to be treated can be easily obtained in displaying a medical image.

The display control device 2 is a display control device 2 configured to make the display 24 display a medical image of a first patient (a patient to be treated) and a medical image of a second patient (another patient who is a patient other than the patient to be treated). The display control device 2 includes the determination unit (controller 21) and the hiding unit (controller 21). The determination unit determines whether to permit displaying the medical image of the second patient on the display 24 in addition to the medical image of the first patient. The hiding unit hides at least one or a plurality of pieces of patient information of the second patient to be shown in the medical image of the second patient in the case in which the determination unit determines to permit displaying the medical image of the second patient.

Thus, it is possible to provide a display control device that enables protecting privacy of a patient other than a patient to be treated, in displaying a medical image.

In the display control device 2, the hiding unit hides at least one or a plurality of pieces of patient information of the second patient to be shown in a manner superimposed on the medical image of the second patient.

Thus, although patient information of another patient is shown superimposed (overlaid) on a medical image of the another patient, privacy of a patient other than a patient to be treated can be protected in displaying a medical image.

In the display control device 2, the hiding unit hides at least one or a plurality of pieces of patient information of the second patient to be embedded in the medical image of the second patient.

Thus, although patient information of another patient is embedded in a medical image of the another patient, privacy of a patient other than a patient to be treated can be protected in displaying a medical image.

The display control device 2 also includes the second obtaining unit that obtains pieces of patient information to be hidden, from among pieces of patient information of the second patient to be shown in a medical image of the second patient.

With this configuration, items that are targets to protect privacy of patient information of a patient other than a patient to be treated can be easily obtained in displaying a medical image.

The medical image display system (system 100) includes the display control device 2, a display device having the display 24 (display control device 2), and the medical image management device (PACS 1) that stores image data of medical images and manages the image data.

Thus, in the medical image display system, privacy of a patient other than a patient to be treated can be protected in displaying a medical image.

Note that the above embodiments describe some examples of a medical image display system, and the present invention is not limited thereto.

For example, in each of step S3 of the patient information concealment process and step S13 of the patient information hiding process of the above-described embodiments, the controller 21 obtains the viewing mode setting information from the storage 23 and determines whether the patient viewing mode is set. However, the processes are not limited thereto. In the state in which the another patient ON mode is set, each of step S3 of the patient information concealment process and step S13 of the patient information hiding process may be omitted, and the controller 21 may conceal or hide at least one or a plurality of pieces of patient information of another patient to be shown in a medical image of the another patient.

In the above-described embodiments, patient information of a patient to be treated is shown in a medical image of the patient to be treated. However, information such as inspection information, series information, or image information, may be shown in addition to the patient information.

In the above-described embodiments, in order to hide patient information of another patient, the contents of the items of patient information to be hidden are shown by asterisks. However, they may be shown by using symbols other than asterisks or by another displaying method.

The display control device 2 of the above-described embodiments may have a function to allow selecting concealing or hiding an item of patient information that is a target to protect privacy of a patient other than a patient to be treated.

Although the controller 21 obtains the concealment setting information from the storage 23 in step S4 of the patient information concealment process of the above-described embodiments, the process is not limited thereto. The controller 21 may obtain concealment setting information that is set and stored in an external device, via the communicator 22.

Although the controller 21 obtains the non-display setting information from the storage 23 in step S14 of the patient information hiding process of the above-described embodiments, the process is not limited thereto. The controller 21 may obtain non-display setting information that is set and stored in an external device, via the communicator 22.

In addition, details of the structure and details of operation of each device that constitutes the medical image display system can also be modified or altered within a range not departing from the gist of the present invention, as appropriate.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A non-transitory computer readable storage medium storing instructions that cause a computer of a display control device to:
determine whether to display, in addition to a medical image of a first patient, a medical image of a second patient on a display; and
upon determining to display the medical image of the second patient, conceal at least one or a plurality of pieces of patient information of the second patient to be shown in the medical image of the second patient.

2. The non-transitory computer readable storage medium according to claim 1, wherein the instructions cause the computer to conceal the patient information of the second patient in a manner superimposed on the medical image of the second patient.

3. The non-transitory computer readable storage medium according to claim 1, wherein the instructions cause the computer to conceal the patient information of the second patient in a manner embedded in the medical image of the second patient.

4. The non-transitory computer readable storage medium according to claim 1, wherein the instructions cause the computer to obtain patient information to be concealed, from among the patient information of the second patient to be shown in the medical image of the second patient.

5. A non-transitory computer readable storage medium storing instructions that cause a computer of a display control device to:
determine whether display, in addition to a medical image of a first patient, a medical image of a second patient on a display; and
upon determining to display the medical image of the second patient, hide at least one or a plurality of pieces of patient information of the second patient to be shown in the medical image of the second patient.

6. The non-transitory computer readable storage medium according to claim 5, wherein the instructions cause the computer to hide the patient information of the second patient in a manner superimposed on the medical image of the second patient.

7. The non-transitory computer readable storage medium according to claim 5, wherein the instructions cause the computer to hide the patient information of the second patient in a manner embedded in the medical image of the second patient.

8. The non-transitory computer readable storage medium according to claim 5, wherein the instructions cause the computer to obtain patient information to be hidden, from among the patient information of the second patient to be shown in the medical image of the second patient.

9. A display control device comprising:
a hardware processor that:
determines whether to display, in addition to a medical image of a first patient, a medical image of a second patient on a display; and
upon determining to display the medical image of the second patient, conceals at least one or a plurality of pieces of patient information of the second patient to be shown in the medical image of the second patient.

10. A display control device comprising:
a hardware processor that:
determines whether to display, in addition to a medical image of a first patient, a medical image of a second patient on a display; and
upon determining to display the medical image of the second patient, hides at least one or a plurality of pieces of patient information of the second patient to be shown in the medical image of the second patient.

11. A medical image display system comprising:
the display control device according to claim 9;
a display device that comprises a display; and
a medical image management device that stores image data of medical images and manages the image data.

12. A medical image display system comprising:
the display control device according to claim 10;
a display device that comprises a display; and
a medical image management device that stores image data of medical images and manages the image data.
